(19) United States
(12) Patent Application Publication
(10) Pub. No.: US 2022/0143079 A1
(43) Pub. Date: May 12, 2022

(54) ORAL ADMINISTRATION OF SODIUM CHLORIDE TO PREVENT COMPLICATIONS ASSOCIATED WITH BOWEL CLEANSING WITH STIMULANT LAXATIVES

(71) Applicant: SEAFORD PHARMACEUTICALS INC., Mississauga (CA)

(72) Inventor: Khursheed N. JEEJEEBHOY, Toronto (CA)

(21) Appl. No.: 17/431,397

(22) PCT Filed: Feb. 18, 2020
(86) PCT No.: PCT/IB2020/000205

§ 371 Date: Aug. 16, 2021

Related U.S. Application Data
(60) Provisional application No. 62/806,023, filed on Feb. 15, 2019.

(51) Int. Cl. A61K 33/14; A61K 31/4402; A61K 31/194; A61K 33/08; A61K 9/00

(52) U.S. Cl. CPC A61K 33/14; A61K 31/4402; A61K 9/0053; A61K 33/08; A61K 31/194

(57) ABSTRACT

A method for preventing unwanted side effects associated with the administration stimulant laxative such as bisacodyl or sodium picosulphate by orally administering sodium chloride to the patient is disclosed. Said side effects include hyponatraemia, hypokalemia, unwanted fluctuations in blood pressure, hypotension and renal failure. In certain embodiments, the sodium chloride is administered in the form of tablets during the two hours period following the administration of the stimulant laxative. This method is particularly useful in preparing patients for a colonoscopy.

### BACKGROUND OF THE INVENTION

Excluding skin cancers, colorectal cancer is the third most common cancer diagnosed in both men and women in the United States. The death rate (the number of deaths per 100,000 people per year) from colorectal cancer, however, has been dropping in both men and women for several decades. There are a number of likely reasons for this. One is that colorectal polyps are now being found more often by screening and removed before they can develop into cancers or are being found earlier when the disease is easier to treat. As a result, there are now more than 1 million survivors of colorectal cancer in the United States alone.

Colonoscopy is the current gold standard when non-invasive methods are positive (i.e., fecal occult blood test, FOBT) in colorectal cancer population screening programs and is also recommended and used as a primary screening modality. Individuals over 50 years of age are recommended to undergo an initial colonoscopy. Subsequent screenings are then scheduled based on the initial results found, with a five- or ten-year recall being common for colonoscopies that produce normal results. Medical societies recommend colonoscopy screenings every 10 years beginning at age 50 for adults without increased risk for colorectal cancer. Colonoscopy reduces cancer rates by detecting some colon polyps and cancers on the left side of the colon early enough that they may be treated, and a smaller number on the right side.

The success of a colonoscopy is largely dependent on the level of bowel cleansing. The colon and rectum must be empty and clean so that the treating physician can see the entire inner lining during the test. Adequate visualization of the colonic mucosa requires a clean colon with no solid or residual brown liquid that could mask a potential lesion. There are different ways to achieve an adequate level of cleanliness, including pills, fluids, and enemas (or combinations of these). For example, a patient might need to drink large amounts of a liquid laxative solution the evening before the procedure and on the morning of the test, sometimes more laxatives or enemas may be needed to make sure the bowels are empty. This often results in a patient spending a lot of time in the bathroom. Because the process of cleaning out the colon and rectum is sometimes unpleasant, it can keep some people from getting this test done.

Bisacodyl is a locally-acting stimulant laxative drug that works directly on the colon to produce a bowel movement in the patient by stimulating the enteric nerves of the muscles in the walls of the small intestine and colon to causing peristalsis. Bisacodyl also alters water and electrolyte levels in the intestine resulting in an increase in the levels of fluids therein which also facilitates bowel movements. As such, it is commonly prescribed as part of a bowel preparation procedure before medical procedures such as a colonoscopy. Bisacodyl is a derivative of triphenylmethane, i.e. ρ,ρ-diacetoxy-diphenyl-2-pyridylmethane, having the following formula:

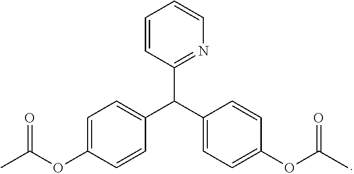 [SMILES: CC(=O)Oc1ccc(C(c2ccc(OC(C)=O)cc2)c2ccccn2)cc1]

Bisacodyl is an OTC drug readily available to a patient in need thereof and sold under several brand names such as Dulcolax®, Durolax, Correctol, Muxol, Fleet®, Nourilx, Purga, Alophen®, Correctol®, Correctol®, Bisacolax, Bisac-Evac, Feen-A-Mint® and Carter's Little Pills®. It is usually sold as 5 mg tablets, 10 mg suppositories or 5 mg or as a 37 ml prepackaged enema containing 10 mg delivered dose of bisacodyl. A dosage of 5 to 30 mg has been prescribed for cleansing of the bowel prior to a colonoscopy or other procedure.

Bisacodyl can cause unwanted fluid and salt secretion (due to the reduction in the resorption of sodium ions and water through inhibition of the sodium and potassium-dependent ATP-ase pathway) resulting in hypotension, hyponatremia, renal failure, abdominal cramping, diarrhea, nausea, vomiting, vertigo, rectal burning and fluid and electrolyte imbalance.

Sodium picosulfate (also known as sodium picosulphate) is also a locally-acting stimulant laxative drug used to prepare the bowel of a patient prior to a colonoscopy procedure. Sodium picosulfate is hydrolysed in the colon and gently stimulates the bowel muscles to increase bowel peristalsis propelling the contents of the bowel out of the body. Sodium picosulfate, is a prodrug of the following formula:

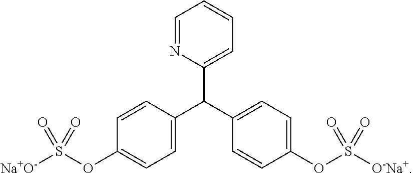

that is metabolized by gastrointestinal flora into the active compound 4,4′-dihydroxydiphenyl-(2-pyridiyl)methane

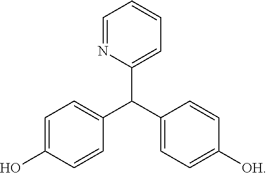 [SMILES: Oc1ccc(C(c2ccc(O)cc2)c2ccccn2)cc1]

Similar to bisacodyl, sodium picosulfate also alters water and electrolyte levels in the intestine. This electrolyte imbalance can lead to hyponatremia and hypokalemia. Increased hemoglobin concentration has also been observed (Barker P et al., (1992), Ann R Coll Surg Engl, 74(5):318-9).

Sodium picosulfate is also an OTC drug sold under the tradenames Sodipic Picofast, Laxoberal, Laxoberon®, Purg-Odan®, Picolax®, Guttalx®, Namilx, Pico-Salax®, PicoPrep Dulcolaz® Pico, CitraFleet® and Prepopik®.

Sodium picosulfate is traditionally administered in combination with magnesium oxide and anhydrous citric acid. Magnesium citrate acts as an osmosis laxative (Cho Y S et al., (2014), Ann Coloproctol, 30(6):290-3). In Canada, powdered forms of each are mixed together at fixed amounts and sold in a small cloth bag (hereinafter referred to as “a sachet”) under the trade name Citracleen® (Recordati S.p.A, Milan, Italy). A Citracleen® sachet contains 10 mg of sodium picosulfate, 10.97 g of citric acid and 3.5 g of magnesium oxide¹ and is dissolved in water prior to oral ingestion. Magnesium oxide and anhydrous citric acid combine when their powder forms are hydrated resulting is magnesium citrate, an osmotic laxative. Both sodium picosulfate and magnesium citrate, when ingested, cause watery diarrhea resulting is the emptying of stool from the colon. ¹Similar products consist of 10 mg sodium picosulfate, 3.5 g magnesium oxide and 12.0 g citric acid combined in a sachet.

It has been observed that a bowel preparation consisting of sodium picosulphate and magnesium citrate had a significant dehydrating effect on patients (Sanders G et al., (2001), Br J Surg, 88(10):1363-5). Sanders et al., confirmed the dehydrating effect of Picolax® bowel preparation in a randomized study assessing the effects of administering intravenous fluid during bowel preparation. One group of patients were administered a bowel preparation of Picolax® and no intravenous fluid while a second group of patients were given a bowel preparation of Picolax® together with a calculated volume of intravenous saline. Sanders et al. observed a significant difference between the groups in mean weight loss, change in erect systolic, erect diastolic and supine diastolic blood pressure, postural blood pressure and serum creatinine concentration as well as a significant fall in erect blood pressure. Sanders et al. opined that the dehydrating effects of a sodium picosulphate and magnesium citrate bowel preparation were minimized by co-administering a volume of intravenous fluid. It should be appreciated that this is an impractical solution.

Several side effects have been observed including hypotension, hyponatremia, renal failure, dehydration, diarrhea, nausea, headache, stomach pain, cramps, dizziness, fainting, loss of consciousness, decreased energy, ocular deviation, decreased urination, bloody stool, seizures, irregular heartbeat, rash, hives and vomiting. In 2005, hyponatremia that resulted in encephalopathy due to the ingestion of sodium picosulfate and magnesium citrate complex was reported (Frizelle F A and Colls B M, (2005), Dis Colon Rectum, 48(2):393-6). A case of hyponatremia accompanied by seizure and loss of consciousness in a patient with normal renal function and no history of seizures after taking a sodium picosulfate and magnesium citrate complex for bowel preparation prior to a colonoscopy was also reported (Cho Y S et al., (2014), Ann Coloproctol, 30(6):290-3).

Both bisacodyl and sodium picosulfate should be used with caution in frail patients (Cho Y S et al., (2014), Ann Coloproctol, 30(6):290-3), patients suffering from hypothyroidism or hypertension, or patients with impaired renal function or inflammatory bowel disease (Weir M A et al., (2014), Am J Gastroenterol, 109(5):686-94) or cardiovascularly compromised patients (Sanders G et al., (2001), Br J Surg, 88(10):1363-5) as both can induce dehydration or electrolyte imbalance (Forde H et al., (2014), Case Rep Clin Med, 3(2):101-4). Various sources discourage the use of either bisacodyl or sodium picosulfate with other laxatives, i.e. in combination (NIH (2018), MedlinePlus, ‘Sodium Picosulfate, Magnesium Oxide, and Anhydrous Citric Acid,” page 3, retrieved from https://medlineplus.gov/druginfo/meds/a613020.html on Sep. 3, 2018). Studies of the purgative effect of bisacodyl and sodium picosulfate in combination resulted in only 52%-80% of patients having an acceptably clean bowel.

The most ideal bowel preparation should not only achieve excellent colon cleansing, but also minimize the adverse impact on the mucosa and changes in the water and the patient's electrolyte levels. In addition, the bowel preparation needs to be drinkable for patient compliance (Rex D K et al., Am J Gastroenterol, 97(7):1696-700), as the intravenous administration of saline is impractical. As such, given the importance of the colonoscopy in the early detection of colon cancer, there is a need for a means in which to prevent the unwanted side effects associated with current colon cleansing regimens, in particular hypotension, hyponatremia and renal failure.

### BRIEF SUMMARY OF THE INVENTION

The instant invention is directed to a method and kit that prevents unwanted side effects associated with traditional bowel cleansing regimens, in particular hypotension, hyponatremia and renal failure.

Another example of the present invention is a bowel cleansing procedure that prevents the side effects caused by the use of traditional stimulant laxative drugs, bisacodyl and sodium picosulfate (or pharmaceutically-acceptable salts or prodrugs thereof)10 alone or in combination with magnesium oxide and citric acid by orally administrating sodium chloride to a patient prior to a bowel cleansing.

A further example of the instant invention is a method of preventing the exacerbation or onset of hyponatremia, hypokalemia, unwanted fluctuation in blood pressure (hypotension) and/or renal failure in patient from the administration of bisacodyl and sodium picosulfate alone or in combination with magnesium oxide and citric acid to a patient to aggressively cleanse their bowel prior to a explorative procedure, such as a colonoscopy.

In yet another embodiment of the instant invention, three 5 mg tablets of bisacodyl, 2 sachets containing a combination of the powder forms sodium picosulfate, magnesium oxide and citric acid and ten 1 mg tablets of sodium chloride to a patient prior to a bowel cleaning event such as a colonoscopy.

In yet a further example, the instant invention is directed to method to cleanse the colon of a patient prior to a colonoscopy without causing unnecessary side effects in particular hypotension, hyponatremia and/or renal failure, comprising the steps of administering orally 3 pre-dosed tablets of bisacodyl with approximately 250 ml of water to a patient in need thereof approximately 24 hours prior to the day said colonoscopy is scheduled; dissolving one sachet containing the sodium picosulfate, magnesium oxide and citric acid combination in approximately 250 ml of water and orally administrating said solution to the patient approximately 2 to 4 hours after the administration of the bisacodyl; administering 1 sodium chloride tablet orally with approximately 250 ml of a clear liquid to the patient every 30 minutes after the administration of said sodium picosulfate, magnesium oxide and citric acid combination for approximately 2 hours; dissolving the second sachet containing the sodium picosulfate, magnesium oxide and citric acid combination in approximately 250 ml of water and orally administrating said solution to the patient approximately 4 to 5 hours before said colonoscopy is scheduled; and administering one sodium chloride tablet orally to the patient every 30 minutes after the administration of said sodium picosulfate, magnesium oxide and citric acid combination for approximately 2 hours.

Another embodiment consists of a kit comprising (i) 3 tablets of bisacodyl; (ii) 2 sachets containing the powder forms of sodium picosulfate, magnesium chloride and citric acid; and (iii) 10 1 g tablets of sodium chloride and optionally (iv) instructions as to the administration of the components of said kit to a patient to cleans the bowel without unwanted side effects of said patient prior to an explorative procedure such as a colonoscopy.

### DETAILED DESCRIPTION OF THE INVENTION

Contrary to teachings of the prior art, it was discovered that bisacodyl (Dulcolax®, Boehringer Ingelheim, Ingelheim am Rhein, Germany) could be administered with a sodium picosulfate/magnesium oxide/citric acid combination (Citracleen®, Recordati S.p.A, Milan, Italy) to improve pre-colonoscopy cleaning of the colon. When administering bisacodyl prior to the ingestion of picosulfate/magnesium oxide/citric acid combination by a patient scheduled for a colonoscopy, the bisacodyl removes the bulk of the stool in the colon whereas the picosulfate/magnesium oxide/citric acid combination effectively removes trace solid feces and/or residual brown liquid, thus improving the quality of the following colonoscopy.

The inventor of the claimed novel method discovered that including the oral ingestion of sodium chloride tablets (Consolidated Midland Corp., Brewster, N.Y.) prevented the onset of blood pressure fluctuation (hypotension), hyponatremia and/or renal failure, as well as obviating a number of side effects observed with the administration of bisacodyl or a sodium picosulfate/magnesium oxide/citric acid combination alone or together. It was discovered that the addition of sodium chloride tablets to a pre-colonoscopy cleansing routine forced patients to drink approximately 2,000 ml (approximately 10 standard drinking glasses) of fluid. It was discovered that the intake of sodium chloride, together with the fluid ingested with said salt dosing, prevented dehydration despite causing watery diarrhea in the patient. Dehydration, which is common side effect caused by the use of stimulant laxatives such as bisacodyl and a sodium picosulfate/magnesium oxide/citric acid combination, can lead to many of the observed, undesirable side effects, and in some cases, particularly in elderly patients and those suffering from renal function issues, can lead to renal failure, an unwanted reduction in blood pressure and an imbalance in electrolytes. The addition of salt tablets to a pre-colonoscopy cleansing regimen maintains hydration, electrolyte balance and normal renal function.

It was discovered that a specific dosing regimen resulted in the best pre-colonoscopy cleansing without the onset of undesirable side effects, a decrease in blood pressure, electrolyte depletion, or decrease in renal function. The preferred embodiment consists of the following colon cleansing regimen:
administering orally 3 tablets of bisacodyl with approximately 250 ml of water to a patient in need thereof approximately 24 hours prior to the day said colonoscopy is scheduled;
dissolving one sachet containing the sodium picosulfate, magnesium oxide and citric acid combination in approximately 250 ml of water and orally administrating said solution to the patient approximately 2 to 4 hours after the administration of the bisacodyl;
administering 1 sodium chloride tablet orally with approximately 250 ml of a clear liquid to the patient every 30 minutes after the administration of said sodium picosulfate, magnesium oxide and citric acid combination for approximately 2 hours;
dissolving the second sachet containing the sodium picosulfate, magnesium oxide and citric acid combination in approximately 250 ml of water and orally administrating said solution to the patient approximately 4 to 5 hours before said colonoscopy is scheduled; and
orally administering one sodium chloride tablet orally to the patient every 30 minutes after the administration of said sodium picosulfate, magnesium oxide and citric acid combination for approximately 2 hours using a clear liquid selected from the group consisting of water, light colored fruit juice without pulp, clear broth, coffee without milk, tea without milk, flavored gelatin, popsicles or soft drinks.

As observed, the initial administration of bisacodyl resulted in an initial expulsion of the bulk of the fecal matter found in the lower large intestine, colon and rectum.

Administration of the sodium picosulfate, magnesium oxide and citric acid combination resulted in the removal of residual stool. The continuous intake of sodium chloride tablets with the accompanying clear liquid maintained the patient's electrolyte levels and renal function, prevented an unwanted decrease in blood pressure, maintained electrolyte levels and prevented dehydration. By maintaining electrolyte levels and keeping the patient hydrated, many of the known side effects cause by stimulant laxative drugs were averted.

Previous studies have investigated the purgative effect of bisacodyl and sodium picosulfate, alone or in combination with a saline purge and a tap water enema (Fork F-T et al., (1982), Gastrointest Radiol, 7(4):383-9). The Fork study involving 1200 patients indicated that the administration of a contact laxative and a saline purge resulted in a “good cleansing” in 52-80% of the patients tested. The Fork study does not address the problems associated with the use of stimulant laxative drugs. Fork discusses use of saline purge with a water enema as a final step to achieve optimum cleansing, however neither the saline purge nor the water enema is sufficient to maintain the electrolyte levels of the patient and only results in further dehydration of the patient. As noted above, the staggered, yet continuous intake of fluid, keeps the patient well-hydrated and the patient's electrolyte levels constant.

### Proof of Concept

A study to determine the effectiveness of the claimed method to prevent the side effects associated with the administration of a stimulant laxative drug to a patient in need of a bowel cleansing prior to a colonoscopy procedure by administering sodium chloride orally to the patient in need thereof was conducted, in particular, whether adding salt tablets with a specific amount of water in addition to the traditional products used for colon cleansing will result in cleaning the colon without dehydration thus causing minimal changes in blood pressure, weight, urine volume and urine sodium.

A prospective, open-label study involving four patients scheduled for their regular colonoscopy exam was conducted (Canadian Phase Onward Inc., Toronto, Ontario. CA). (The original study included five patients, however, the subject identified as Patient #1 failed to follow the protocol and was dropped from the study. The selected patients were over the age of 18 and all consented to be part of the study.) After a patient was included in the study, their weight, age, sex, ethnicity, and blood pressure was measured and recorded. In addition to the standard instructions not to eat food and to drink only clear liquids, the selected patients were instructed to do the following on the day before the colonoscopy was to be performed:

1. at 10:00 AM orally taking three bisacodyl pills (Dulcolax®, Boehringer Ingelheim, Ingelheim am Rhein, Germany) with water (chewing or crushing the pills was prohibited);

2. at 3:00 PM dissolving the contents of a first sachet containing sodium picosulfate/magnesium oxide/citric acid combination (Citracleen®, Recordati S.p.A, Milan, Italy) with cold water and ingesting the solution after carefully stirring the solution to insure that the sodium picosulfate/magnesium oxide/citric acid combination was dissolved completely;

3. ingesting approximately 250 ml of a clear fluid with a 1 g sodium chloride tablet every half hour after ingestion of the sodium picosulfate/magnesium oxide/citric acid combination solution;

4. repeating step 3 every half hour until five (5) salt tablets have been ingested over a two and half hour period;

5. at approximately five hours prior to the time that the patient's colonoscopy is scheduled, dissolving the contents of a second sachet containing sodium picosulfate/magnesium oxide/citric acid combination (Citracleen®, Recordati S.p.A, Milan, Italy) with cold water and ingesting the solution after carefully stirring the solution to insure that the sodium picosulfate/magnesium oxide/citric acid combination was dissolved completely; and

6. repeating steps 3 and 4 until five (5) salt tablets have been ingested over a two and half hour period.

Upon arrival at the clinic on the day of the colonoscopy, the patient's weight was recorded and blood pressure was taken and recorded and an out-patient colonoscopy was performed on each patient (North York Endoscopy Centre Inc., Toronto, Ontario, CA). The quality of the colon cleaning was assessed using the Boston Scale which is a well-established way of identifying the degree of colon cleanliness.

After the colonoscopy was completed, each patient was given a urine collection container and instructed to collect their urine for the following twenty four (24) hours and return the collected urine to the clinic for analysis.

### Patient Case Studies

Patient #2 is a 72 year old Caucasian male with a medical history of diverticulitis, arthritis, sleep apnea, fatty live disease and distal abnormal aortic ectasia as well as having epidermal cysts removed and surgery on the patient's right hand. The patient takes a daily multi-vitamin as well as Advil® PM for body aches and pains. He was diagnosed as needing a colonoscopy by his treating physician. At the time of information intake, the patient's weight was recorded as 109.5 kg and his blood pressure recorded at 155/90 mmHg. The patient was given bowel cleaning preparation medications: three bisacodyl pills (Dulcolax®, Boehringer Ingelheim, Ingelheim am Rhein, Germany)), two sachets of picosulfate/magnesium oxide/citric acid combination (Citracleen®, Recordati S.p.A, Milan, Italy) and ten 1 g NaCl tablets, as well as instructions to follow the claimed bowel cleansing method as set forth above.

On the day of the scheduled colonoscopy, the patient confirmed that he followed the instructions provided and ingested all of the proper amounts of the products given to him at the appropriate times as set forth in the instructions provided to the patient. The patient's blood pressure was taken and recorded as 145/80 mmHg and his weight was recorded at 160.4 kg. After completion of the colonoscopy, the Boston Bowel Preparation score was evaluated at a 9. No adverse events were observed.

The patient was given a urine collection container and instructed to collect his urine for the twenty four (24) hour period following the colonoscopy and return the collected urine to the clinic for analysis. The results of the analysis of Patient #2's urinalysis is presented in Table 1:

TABLE 1
Patient #2 Urine Analysis Results

| | | | REFER- | | |
|---|---|---|---|---|---|
| TEST NAME | | | ENCE | | TEST |
| 24 HR URINE | RESULT | FLAG | RANGE | UNITS | LOC. |
| TOTAL VOLUME | 2.80 | | | L/day | 10 |
| CREATININE | 6.2 | | 3.5-24.5 | mmol/L | |
| CONCENTRATION | | | | | |
| CREATININE | 17.4 | | 7.8-20.0 | mmol/ | |
| PER DAY | | | | day | |
| SODIUM | 65 | | | mmol/L | |
| CONCENTRATION | | | | | |
| SODIUM PER DAY | 182 | | 40-220 | mmol/ | |
| | | | | day | |

Patient #3 is a 45 year old African American female with a medical history of diabetes mellitus type IL, hyperthyroidism, obstructive sleep apnea, fatty liver disease, osteoarthritis of the right knee as well as undergoing a thyroidectomy, oophorectomy and a total hysterectomy. The patient also reported abdominal pain and melena on occasion. She takes 1000 mg metformin and 0.175 levothyroxine daily. She was diagnosed as needing a colonoscopy by her treating physician. At the time of information intake, the patient's weight was recorded as 97.0 kg and her blood pressure recorded at 180/100 mmHg. The patient was given bowel cleaning preparation medications: three bisacodyl pills (Dulcolax®, Boehringer Ingelheim, Ingelheim am Rhein, Germany)), two sachets of picosulfate/magnesium oxide/citric acid combination (Citracleen®, Recordati S.p.A, Milan, Italy) and ten 1 g NaCl tablets, as well as instructions to follow the claimed bowel cleansing method as set forth above.

On the day of the scheduled colonoscopy, the patient confirmed that she followed the instructions provided and ingested all of the proper amounts of the products given to her at the appropriate times as set forth in the instructions provided to the patient. The patient's blood pressure was taken and recorded as 150/99 mmHg and her weight was recorded at 97.0 kg. After completion of the colonoscopy, the Boston Bowel Preparation score was evaluated at a 9. No adverse events were observed.

The patient was given a urine collection container and instructed to collect her urine for the twenty four (24) hour period following the colonoscopy and return the collected urine to the clinic for analysis. The results of the analysis of Patient #2's urinalysis is presented in Table 2:

TABLE 2
Patient #3 Urine Analysis Results

| | | | REFER- | | |
|---|---|---|---|---|---|
| TEST NAME | | | ENCE | | TEST |
| 24 HR URINE | RESULT | FLAG | RANGE | UNITS | LOC. |
| TOTAL VOLUME | 1.00 | | | L/day | 10 |
| CREATININE | 16.0 | | 2.5-20.0 | mmol/L | |
| CONCENTRATION | | | | | |
| CREATININE | 16.0 | | 5.5-17.5 | mmol/ | |
| PER DAY | | | | day | |
| SODIUM | 194 | | | mmol/L | |
| CONCENTRATION | | | | | |
| SODIUM PER DAY | 194 | | 40-220 | mmol/ | |
| | | | | day | |

Patient #4 is a 28 year old Caucasian male with a medical history of abdominal pain, left varicocele, genital warts and an allergy to eggs as well as having planter warts removed and acromioclavicular joint surgery. The patient did not report taking any concomitant medications. He was diagnosed as needing a colonoscopy by his treating physician. At the time of information intake, the patient's weight was recorded as 79.0 kg and his blood pressure recorded at 120/70 mmHg. The patient was given bowel cleaning preparation medications: three bisacodyl pills (Dulcolax®, Boehringer Ingelheim, Ingelheim am Rhein, Germany)), two sachets of picosulfate/magnesium oxide/citric acid combination (Citracleen®, Recordati S.p.A, Milan, Italy) and ten 1 g NaCl tablets, as well as instructions to follow the claimed bowel cleansing method as set forth above.

On the day of the scheduled colonoscopy, the patient confirmed that he followed the instructions provided and ingested all of the proper amounts of the products given to him at the appropriate times as set forth in the instructions provided to the patient. The patient's blood pressure was taken and recorded as 121/74 mmHg and his weight was recorded at 79.2 kg. After completion of the colonoscopy, the Boston Bowel Preparation score was determined to be a 10. No adverse events were observed.

The patient was given a urine collection container and instructed to collect his urine for the twenty four (24) hour period following his colonoscopy and return the collected urine to the clinic for analysis. The results of the analysis of Patient #4's urinalysis is presented in Table 3:

TABLE 3
Patient #4 Urine Analysis Results

| | | | REFER- | | |
|---|---|---|---|---|---|
| TEST NAME | | | ENCE | | TEST |
| 24 HR URINE | RESULT | FLAG | RANGE | UNITS | LOC. |
| TOTAL VOLUME | 1.60 | | | L/day | 10 |
| CREATININE | 9.2 | | 3.5-24.5 | mmol/L | |
| CONCENTRATION | | | | | |
| CREATININE | 14.7 | | 7.8-20.0 | mmol/ | |
| PER DAY | | | | day | |
| SODIUM | 37 | | | mmol/L | |
| CONCENTRATION | | | | | |

Test repeated and results confirmed.

| | | | |
|---|---|---|---|
| SODIUM PER DAY | 59 | 40-220 | mmol/ |
| | | | day |

Patient #5 is a 58 year old Caucasian female with a medical history of constipation, hemorrhoids, recurrent rectal bleeding, recurrent colonic polyps, fatty liver disease, osteopenia, osteoporosis, dyslipidemia, anxiety, varicose veins and diabetes mellitus type II. The patient takes 90 mgs of duloxetine daily and Dulcolax® as needed. She was diagnosed as needing a colonoscopy by her treating physician. At the time of information intake, the patient's weight was recorded as 65.6 kg and her blood pressure recorded at 130/75 mmHg. The patient was given bowel cleaning preparation medications: three bisacodyl pills (Dulcolax®, Boehringer Ingelheim, Ingelheim am Rhein, Germany)), two sachets of picosulfate/magnesium oxide/citric acid combination (Citracleen®, Recordati S.p.A, Milan, Italy) and ten 1 g NaCl tablets, as well as instructions to follow the claimed bowel cleansing method as set forth above.

On the day of the scheduled colonoscopy, the patient confirmed that she followed the instructions provided and ingested all of the proper amounts of the products given to her at the appropriate times as set forth in the instructions provided to the patient. The patient's blood pressure was taken and recorded as 114/66 mmHg and her weight was recorded at 66.4 kg. After completion of the colonoscopy, the Boston Bowel Preparation score was determined to be a 9. No adverse events were observed.

The patient was given a urine collection container and instructed to collect her urine for the twenty four (24) hour period following her colonoscopy and return the collected urine to the clinic for analysis. The results of the analysis of Patient #5's urinalysis is presented in Table 4:

TABLE 4
Patient #5 Urine Analysis Results

| | | | REFER- | | |
|---|---|---|---|---|---|
| TEST NAME | | | ENCE | | TEST |
| 24 HR URINE | RESULT | FLAG | RANGE | UNITS | LOC. |
| TOTAL VOLUME | 1.00 | | | L/day | 10 |
| CREATININE | 6.1 | | 2.5-20.0 | mmol/L | |
| CONCENTRATION | | | | | |
| CREATININE | 6.1 | | 5.5-17.5 | mmol/ | |
| PER DAY | | | | day | |
| SODIUM | 64 | | | mmol/L | |
| CONCENTRATION | | | | | |
| SODIUM PER DAY | 64 | | 40-220 | mmol/ | |
| | | | | day | |

### Kits

This disclosure also provides kits for conveniently and effectively implementing the methods disclosed herein. Such kits comprise any subject composition, and a means for facilitating compliance with methods disclosed herein. Such kits provide a convenient and effective means for assuring that the subject to be treated self-administers the appropriate active in the correct dosage in the correct manner. The compliance means of such kits includes any means which facilitates administering the actives according to a method disclosed herein. Such compliance means include instructions, packaging, and dispensing means, and combinations thereof. Kit components may be packaged for either manual or partially or wholly automated practice of the foregoing methods. In other embodiments involving kits, the disclosure contemplates a kit including compositions disclosed herein, and optionally instructions for their use.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%.

The above discussion is meant to be illustrative of the principle and various embodiments of the present invention. Numerous variations and modifications will become apparent to those skilled in the art once the above disclosure is fully appreciated. It is intended that the following claims be interpreted to embrace all such variations and modifications.

What is claimed:

1. A method to prevent the side effects associated with the administration of a stimulant laxative drug by administering sodium chloride orally to the patient in need thereof to whom said stimulant laxative drug is administered.

2. The method according to claim 1, wherein said stimulant laxative drug is a derivative of triphenylmethane.

3. The method according to claim 2, wherein said stimulant laxative drug selected from the group consisting of ρ,ρ-diacetoxy-diphenyl-2-pyridylmethane, 4,4′-dihydroxydiphenyl-(2-pyridyl)methane, pharmaceutically acceptable salts thereof, prodrugs thereof and any combination thereof.

4. The method according to claim 3, wherein said ρ,ρ-diacetoxy-diphenyl-2-pyridylmethane is bisacodyl.

5. The method according to claim 3, wherein said prodrug of 4,4′-dihydroxydiphenyl-(2-pyridyl)methane is sodium picosulfate.

6. The method according to claim 1, wherein the patient is administered a combination of bisacodyl and sodium picosulfate.

7. The methods according to claims 1, 3 or 6 wherein said patient is administered 10 g of sodium chloride.

8. The method according to claim 7, wherein said patient is administered 15 mg of bisacodyl.

9. The method according to claim 7, wherein said patient is administered 10 mg of sodium picosulfate.

10. The method according to claim 7, wherein the patient is administered said sodium chloride in incremental doses.

11. The method according to claim 10, wherein said patient is administered ten 1 g tablets of sodium chloride.

12. The method according to claim 11, wherein said patient administers said tablets to themselves upon the advice of a treating technician.

13. The method according to claim 5, further comprising administering magnesium oxide and citric acid with said sodium picosulfate.

14. The method according to claim 13, wherein said sodium picosulfate, magnesium oxide and citric acid are in the form of dry ingredients mixed together and contained in a sachet for administration to the patient in need thereof by the patient.

15. The method according to claim 14, wherein said sachet contains 10 mg of sodium picosulfate, 3.5 g of magnesium oxide and approximately 10-12 g of citric acid.

16. The method according to claim 14, wherein two sachets sodium picosulfate, magnesium oxide and citric acid are in the form of dry ingredients are administered to the patient in need thereof.

17. The method of claim 16, wherein said sachets are dissolved in water prior to administration.

18. The method of claim 17, wherein said sachet is dissolved in approximately 250 ml of water.

19. The method according to claim 7, wherein said bisacodyl is a pre-dosed tablet.

20. The method according to claim 19, wherein 3 tablets of bisacodyl are administered to a patient in need thereof.

21. The method according to claim 7, wherein said patient is in need of a bowel cleaning prior to a diagnostic procedure requiring a clean bowel.

22. The method according to claim 19, wherein said diagnostic procedure is a colonoscopy.

23. The method according to claim 7, wherein 3 tablets of bisacodyl, 2 sachets of a combination of sodium picosulfate, magnesium oxide and citric acid and 10 tablets of sodium chloride are administered to a patient in need thereof.

24. The method according to claim 23, comprising the steps of
administering orally 3 tablets of bisacodyl with approximately 250 ml of water to a patient in need thereof approximately 24 hours prior to the day said colonoscopy is scheduled;
dissolving one sachet containing the sodium picosulfate, magnesium oxide and citric acid combination in approximately 250 ml of water and orally administrating said solution to the patient approximately 2 to 4 hours after the administration of the bisacodyl;
administering 1 sodium chloride tablet orally with approximately 250 ml of a clear liquid to the patient every 30 minutes after the administration of said sodium picosulfate, magnesium oxide and citric acid combination for approximately 2 hours;
dissolving the second sachet containing the sodium picosulfate, magnesium oxide and citric acid combination in approximately 250 ml of water and orally administrating said solution to the patient approximately 4 to 5 hours before said colonoscopy is scheduled; and
administering one sodium chloride tablet orally to the patient every 30 minutes after the administration of said sodium picosulfate, magnesium oxide and citric acid combination for approximately 2 hours.

25. The method according to claim 24, wherein said clear liquid is selected from the group consisting of water, light colored fruit juice without pulp, clear broth, coffee without milk, tea without milk, flavored gelatin, popsicles and soft drinks.

26. The method according to claim 1, wherein said method prevents hyponatremia, hypokalemia, unwanted fluctuation in blood pressure, hypotension and/or renal failure.

27. The method according to claim 1, wherein said side effects are selected from the group consisting of dehydration, diarrhea, dizziness, ocular deviation, decreased urination, bloody stool, seizures, irregular heartbeat, rash, hives, nausea and vomiting, headache, short-term memory loss, confusion, lethargy, fatigue, loss of appetite, irritability, muscle weakness, spasms or cramps, vertigo, fluid and electrolyte imbalance, decreased consciousness, abdominal pain, fainting and any combination thereof.

28. The method according to any one of claims 24 to 26, wherein the patient is elderly or suffering from renal failure.

29. A kit comprising (i) 3 tablets of bisacodyl; (ii) 2 sachets containing the powder forms of sodium picosulfate, magnesium chloride and citric acid; and (iii) 10 1 g tablets of sodium chloride.

30. The kit according to claim 29, further comprising (iv) instructions as to the administration of the components of said kit to a patient to cleanse the bowel of said patient prior to a colonoscopy procedure.

31. The kit according to claim 29, wherein when the contents of said kit are used to cleanse the bowel of a patient prior to a colonoscopy procedure.
